(19) United States
(12) Patent Application Publication
(10) Pub. No.: US 2016/0038542 A1
(43) Pub. Date: Feb. 11, 2016

(54) TREATMENT OF INSULIN RESISTANCE AND DIABETES

(71) Applicant: Creative Medical Health Inc., Phoenix, AZ (US)

(72) Inventors: Neil H. Riordan, trophy club, TX (US); Tom Ichim, San Diego, CA (US)

(21) Appl. No.: 14/463,572

(22) Filed: Aug. 19, 2014

Related U.S. Application Data
(63) Continuation of application No. 12/108,114, filed on Apr. 23, 2008.
(60) Provisional application No. 60/913,533, filed on Apr. 23, 2007.

(51) Int. Cl. A61K 35/28

(52) U.S. Cl. CPC A61K 35/28

(57) ABSTRACT

Disclosed are methods, compositions, and cells useful for increasing insulin sensitivity, as well as lack of insulin production in a host in need thereof. One aspect of the invention discloses methods of increasing skeletal muscle perfusion through administration of cells capable of directly and/or indirectly stimulatory of angiogenesis and/or vascular responsiveness. Another aspect provides means of increasing sensitivity to insulin through administration of a cell composition capable of integrating into host insulin responsive tissue and upregulating responsiveness either through mobilization of host cells capable of responding to insulin, mobilization of host cells capable of endowing insulin responsiveness on other host cells, exogenously administered cells taking the role of insulin responsiveness, or exogenously administered cells endowing insulin responsiveness on other host cells. Another aspect comprises modifying said host to allow for concurrent insulin sensitization and upregulated production of insulin.

### CROSS-REFERENCE TO RELATED APPLICATIONS

This application claims priority to Provisional Application Ser. No. 60/913,533, filed Apr. 23, 2007, entitled “Treatment of Insulin Resistance and Diabetes”, and is a continuation of application Ser. No. 12/108,114, filed Apr. 23, 2008, each of which is hereby expressly incorporated by reference in its entirety.

### FIELD OF THE INVENTION

The invention relates to the field of metabolic diseases. Particularly the invention discloses methods of treating insulin resistance and providing an environment suitable for restoration of insulin producing cell function. More particularly the invention relates to methods of treating insulin resistance using cell therapy and combinations of cell therapy with various pharmacological and medical interventions.

### BACKGROUND

Diabetes is a disease of hyperglycemia. There are two main forms of diabetes, Type 1 diabetes, and Type 2. In Type 1 diabetes, also known as insulin-dependent diabetes mellitus (IDDM), or juvenile diabetes, the patient's pancreas produces little or no insulin, believed to be in part the result of autoimmune attached on the insulin producing beta-cells in the pancreas. It's one of the most costly, chronic diseases of childhood and one you never outgrow. It is believed that more than one million Americans have IDDM. Patients with full-blown IDDM must take multiple insulin injections daily or continually infuse insulin through a pump, and test their blood sugar by pricking their fingers for blood six or more times per day. Neither dietary therapy nor treatment with an oral hypoglycemic agent is effective, and only treatment with insulin is effective. Ketonemia and acidosis due to the loss of insulin secreting capacity, and if untreated, may result in diabetic coma. Since numerous factors such as stress, hormones, growth, physical activity, medications, illness/infection, and fatigue effect insulin utilization, even a strictly monitored program of insulin administration does not mimic the endogenous functions of the pancreas, and as a result numerous complications develop.

Type 2 diabetes, also known as Non-Insulin Dependent Diabetes Mellitus (NIDDM), or adult-onset diabetes, is associated with impairment of peripheral tissue response to insulin. NIDDM is believed to afflict approximately 18.2 million people in the US and as a result of the obesity epidemic, substantially younger patients are beginning to be diagnosed with this condition. The economic burden of NIDDM is witnessed in statistics demonstrating that on average, the health care costs for NIDDM patients are approximately $13,243 for people with NIDDM, whereas age-matched controls is $2560 per year.

Insulin resistance is present in almost all obese individuals (1). However, compensatory insulin production by beta-cells usually occurs, thus preventing hyperglycemia. In response to prolonged insulin resistance, as well as other factors, beta cell insulin production eventually lose ability to cope with the increasing insulin demands and postprandial hyperglycemia occurs, characterizing the transition between normal glucose tolerance and abnormal glucose tolerance. Subsequently, the liver starts secreting glucose through hepatic gluconeogenesis (generation of glucose from substrates that are not sugars, not from glycogen) and hyperglycemia is observed even in the fasting state. In contrast to IDDM, NIDDM presents only a small degree of ketonemia and acidosis although the insulin action is reduced from normal, and treatment with insulin is not always required.

The greatest clinical challenge in this disease is the prevention of the long-term complications, many of which involve vascular, ocular and renal systems. Although various agents are utilized to increase glucose sensitivity, insulin secretion, or exogenous insulin is used therapeutically, these do not exactly mimic the physiological control of post-prandial insulin secretion. Accordingly, the fluctuations of glucose, as well as downstream metabolic consequences end up causing macrovascular pathology such as coronary atherosclerosis, and increased risk of stroke, as well as microvascular pathology such as macular degeneration and renal failure. Additionally, neuropathies are often present associated with hyperglycemia.

There are numerous treatments available for NIDDM; these depend on patient-specific characteristics, as well as severity of disease. The treatment goal in diabetes treatment is to bring plasma glucose levels down to as near normal levels, for example 80-120 milligrams per deciliter (mg/dl) before meals and 100-140 mg/dl at night. Numerous medical tests are known in the art for monitoring glucose, as well as cholesterol and lipid levels. The goal of maintaining normal glucose levels is judged in some ways, by the ability to prevent secondary complications such as retinopathy, neuropathy, vascular disease, and strokes.

In beginning phases of NIDDM patients may be treated with various oral drugs, as diabetes progresses, various forms of insulin may be administered. Although tight glucose control is known to decrease the rate of diabetic complications, such control is very difficult to achieve, and when achieved significant morbidity and mortality still occurs. Below are listed some of the non-insulin treatments for NIDDM.

Mainstream oral treatments for diabetes can be separated by mechanism of action into two groups: hypoglycemics, such as sulfonylureas and meglitinides which induce beta cell insulin secretion and antihyperglycemics such as biguanides and alpha-glucosidase inhibitors which cause uptake of glucose.

Sulfonylureas are a type of drug that stimulate insulin release from beta cells. Essentially, these agents work by blocking ATP-sensitive potassium channels in the pancreatic beta-cell membrane. This effect is mediated by the binding of the drug to the sulfonylurea receptor (SUR) subunit of the channel. Inhibition of the potassium channel leads to depolarization of the cell membrane and insulin secretion, in a similar way as if glucose was added to the cell. Glyburide is a second generation sulfonylurea compound that is sold under the names Micronase, DiaBeta, or Glynase. Glipizide, sold under the names Glucotrol and Glucotrol XL, is also a second generation sulfonylurea drug. Third-generation sulfonylurea drugs include Glimepiride (Amaryl). This agent is believed to have greater safety in patients with ischemic heart disease as compared to other sulfonylurea drugs. Glimepiride is the only sulfonylurea based drug that is approved for use together with insulin or metformin. In general, sulfonylurea drugs suffer from the disadvantage that the amount of insulin secretion induced depends on the timing and dose of drug administration and not by the blood glucose levels. This causes not only various fluctuations in glucose level but also digestive symptoms such as anorexia in some patients.

Meglitinides (commonly called glinides) are a class of insulin secretagogues that have short-acting activity, given after meals. Similar to sulfonylurea drugs in that mechanistically they induce insulin secretion by closure of the ATP-dependent potassium channel, glinides appear to be more short-term in activity. Theoretically these drugs have less risk of inducing hypoclycemia and cause a physiological-like insulin release pattern. Repaglinide, sold under the name Prandin, and Nateglinide, sold under the name Starlix, are examples of two glinides. When compared with sulfonylurea drugs, glinides have been shown to provide a better control of postprandial hyperglycaemia, not to induce hypoglycaemia, and to generally have better safety profile, especially in patients with renal failure (2).

Biguanides are a class of drugs that decrease hepatic glucose production and increase insulin sensitivity. Metformin, sold under the names Glucophage, Glucophage XR, and Metformin XR is an example of a biguanide. It is also the most widely prescribed oral antidiabetic in the world and is in most circumstances the agent of choice for first line initial therapy of the typical obese patient with type 2 DM and mild to moderate hyperglycaemia (3). Metformin administration is associated with weight loss and improvement in lipid profile. Metformin is effective as monotherapy and, in combination with both insulin secretagogues and thiazolidinediones (TZDs), may alleviate the need for insulin treatment (4). It is known that metformin induces increased glucose utilization and reduction in leptin concentrations (5). Additionally, metformin induces inhibition of dipeptidyl peptidase-IV activity, which allows for extended half-life of GLP-1 (6). Classical mechanisms of action include increased glucose use by anaerobic glycolysis, inhibition of hepatic gluconeogenesis, and suppression of intestinal absorption of glucose. One adverse effect associated with various biguanides is lactic acidosis.

Thiazolidinediones (glitazones) are a family of drugs that decrease insulin resistance in both muscle and adipose tissue. They do not induce insulin secretion. Rosiglitazone, sold under the name Avandia, and Pioglitazone, sold under the name Actos are two thiazolidinediones. These agents induce insulin sensitivity through the activation of insulin receptor kinase, thereby promoting glucose uptake by peripheral tissues, and ameliorating increased liver glucose production. Known side effects include digestive symptoms and edema, and hematological alterations, and upregulation in plasma LDH. Glitazones are interesting not only from their ability to increase insulin signal transduction, but also due to anti-inflammatory effects. It is known, for example, that rosiglitazone inhibits ability of dendritic cells to secrete interleukin-12 after stimulation via CD40 (7). This is believed to occur via activation of PPAR-gamma pathways. Additionally, treatment with rosiglitazone is able to inhibit onset of colitis in animal models through preferential induction of Th2 cytokine production (8).

Alpha-glucosidase inhibitors are used to delay rate of sugar absorption. Acarbose, sold under the name Precose, and Miglitol sold under the name Glyset are two examples of drugs in this family.

Incretin mimetics mirror glucose-dependent insulin secretion, cause inhibition of glucagon secretion, and delay gastric emptying. Exenatide, sold under the name Byetta, is a glucagon-like-peptide-1 (GLP-1) receptor agonist and stimulates insulin secretion from the beta cell. Controlled clinical trials provided evidence that glycaemic control under exenatide administered twice daily in a dose of 5 - 10 microg was not inferior to conventional insulin therapy.

It is apparent from the prior art that currently available treatments for NIDDM lack the capability of mimicking an endogenous insulin secretion and insulin utilization response. Accordingly various approaches have been pursued aimed at utilization of cell therapy for generating synthetic islets. These approaches have included U.S. Pat. No. 7,056,734 which discloses the ability of GLP and Exendin-4 to induce differentiation of cells into insulin produce or amylase producing cells. The patent covers use of GLP-1 or related molecules to make either non-insulin producing cells, or amylase producing cells, into insulin producing cells, as well as using Exendin-4 for making either non-insulin producing cells, or amylase producing cells, into insulin producing cells.

U.S. Pat. No. 6,903,073 covers the stimulation of hedgehog expression to increase insulin production. This is based on findings that inhibiting hedgehog signaling reduces insulin production, and transfection with hedgehog increases insulin production (9).

U.S. Pat. No. 6,967,019 discloses ways of making gastrointestinal organ cells and pancreatic cells express insulin in vitro, conceptually for introduction in vivo. The patent essentially teaches that introduction of a neuroendocrine class B basic helix-loop-helix (bHLH) transcription factor gene or the neurogenin3 (Ngn3) gene into gastrointestinal organ cells or pancreatic cells, respectively, endows ability to produce insulin. Unfortunately, no evidence of glucose regulation was provided.

U.S. Pat. No. 7,033,831 teaches method of generating insulin producing cells from human embryonic stem cells through the process of first incubating the human embryonic stem cells with Activin A, and then subsequently incubating the cells with nicotinamide. Activin is a peptide involved in wound healing and morphogenesis, whereas nicotinamide is a type of vitamin B3 and improves beta cell functions. The patent covers the culturing of ES cells first in Activin A, then nicotinamide as a method of generating insulin producing cells. Also covered are methods of producing insulin secreting cells, through first growing embryoid bodies, then treating the embryoid bodies with a TGF-b antagonist together with one or more mitogens (to stimulate proliferation), and subsequently culturing the cells in nicotinamide. Additionally covered is the use of embryonic stem cells and not embryoid bodies as starting tissue for generation of insulin producing cells.

U.S. Pat. No. 7,169,608 describes a simple method of inducing differentiation of bone marrow into islets by a simple two step culture approach involving an initial culture in low concentration of glucose (at least 3 days) followed by a subsequent culture in high concentration of glucose (at least 7 days). According to the patent, the resulting cells generate insulin in response to sugar, and are capable of preventing diabetes when administered in vivo into animals. The patent is interesting because authors have actually published some of the data from the patent (10). Noteworthy points about the published data is that the bone marrow derived cells appear to take an architecture similar to that found in the normal islets when administered in vivo. The transplanted cells produce insulin (I and II), glucagon, somatostatin and pancreatic polypeptide, and C-peptide. In addition, various animal models of diabetes were cured by administration of bone marrow cells that were manipulated according to the invention.

U.S. Pat. No. 7,138,275 teaches that culturing of peripheral blood monocytes in the presence of IL-3 and M-CSF for approximately 6 days, induces a program of de-differentiation in the monocytes to endow them with stem cell like potential. The patent goes on to demonstrate that these monocytes can be converted into islets, and shows efficacy in a streptozocin-treated diabetic mouse model of diabetes.

For the above patents it is obvious that although some generation of insulin producing cells was reported in vitro, and in some cases, in vivo, therapeutic applications of this is limited. In NIDDM, the high insulin demands needed to overcome insulin resistance place significant stress on the beta cell. This “need” for hyperinsulin production, as well as other factors associated with hyperglycemia often lead to accelerated beta cell apoptosis through mechanisms such as Fas, the ATP-sensitive K+ channel, insulin receptor substrate 2, oxidative stress, nuclear factor-kappaB, endoplasmic reticulum stress, and mitochondrial dysfunction (11). Thus even if an appropriate beta cell source could be generated as described in the above patents, it is unlikely to yield long-term beneficial clinical results due to the underlying causative elements that initiated diabetes onset originally.

### SUMMARY OF THE INVENTION

The invention provides methods of increasing insulin sensitivity in a mammal through administration of a cell population capable of augmenting perfusion of skeletal muscles, said insulin resistance may be caused by a number of factors including diabetes, aging, low grade inflammation, obesity, pregnancy, metabolic syndrome X, and congenital abnormality. In one aspect, the invention provides cells capable of stimulating perfusion of skeletal muscles, which may be administered systemically or locally into said skeletal muscle with the aim of increasing blood flow.

In one aspect cells capable of stimulating perfusion may be selected from a group comprising of: stem cells, committed progenitor cells, and differentiated cells. Said stem cells may be selected from a group comprising of: embryonic stem cells, cord blood stem cells, placental stem cells, bone marrow stem cells, amniotic fluid stem cells, neuronal stem cells, circulating peripheral blood stem cells, mesenchymal stem cells, germinal stem cells, adipose tissue derived stem cells, exfoliated teeth derived stem cells, hair follicle stem cells, dermal stem cells, parthenogenically derived stem cells, reprogrammed stem cells and side population stem cells. In some aspects of the invention, embryonic stem cells are totipotent and may express one or more antigens selected from a group consisting of: stage-specific embryonic antigens (SSEA) 3, SSEA 4, Tra-1-60 and Tra-1-81, Oct-3/4, Cripto, gastrin-releasing peptide (GRP) receptor, podocalyxin-like protein (PODXL), Rex-1, GCTM-2, Nanog, and human telomerase reverse transcriptase (hTERT). Non-embryonic stem cells may be derived from cord blood stem cells possess multipotent properties and are capable of differentiating into endothelial, smooth muscle, and neuronal cells. Cord blood stem cells useful for the practice of the invention may be identified based on expression of one or more antigens selected from a group comprising: SSEA-3, SSEA-4, CD9, CD34, c-kit, OCT-4, Nanog, and CXCR-4, additionally, cord blood stem cells do not express one or more markers selected from a group comprising of: CD3, CD34, CD45, and CD11b.

In another aspect of the invention, placental stem cells are isolated from the placental structure and administered for the purpose of increasing perfusion of skeletal muscles. Said placental stem cells are identified based on expression of one or more antigens selected from a group comprising: Oct-4, Rex-1, CD9, CD13, CD29, CD44, CD166, CD90, CD105, SH-3, SH-4, TRA-1-60, TRA-1-81, SSEA-4 and Sox-2.

In another aspect of the invention, bone marrow stem cells are isolated from the bone marrow and administered for the purpose of increasing perfusion of skeletal muscles. Said bone marrow stem cells may be bone marrow derived mononuclear cells, said mononuclear cells containing populations capable of differentiating into one or more of the following cell types: endothelial cells, smooth muscle cells, and neuronal cells. In one embodiment, said bone marrow stem cells may be selected based on expression of one or more of the following antigens: CD34, c-kit, flk-1, Stro-1, CD105, CD73, CD31, CD146, vascular endothelial-cadherin, CD133 and CXCR-4. Additionally, stem cell activity may be enhanced by selecting for cells expressing the marker CD133.

In another aspect of the invention, stem cells may be isolated from amniotic fluid and used for stimulation of skeletal muscle perfusion. Said isolation may be accomplished by purifying mononuclear cells, and/or c-kit expressing cells from amniotic fluid, said fluid may be extracted by means known to one of skill in the art, including utilization of ultrasound guidance. Said amniotic fluid stem cells may be selected based on expression of one or more of the following antigens: SSEA3, SSEA4, Tra-1-60, Tra-1-81, Tra-2-54, HLA class I, CD13, CD44, CD49b, CD105, Oct-4, Rex-1, DAZL and Runx-1 or lack of significant expression of one or more of the following antigens: CD34, CD45, and HLA Class II.

In another aspect of the invention, neuronal stem cells may be utilized as a cell source capable of stimulating perfusion of skeletal muscle. Said neuronal stem cells are selected based on expression of one or more of the following antigens: RC-2, 3CB2, BLB, Sox-2hh, GLAST, Pax 6, nestin, Muashi-1, NCAM, A2B5 and prominin.

In another aspect of the invention, circulating peripheral blood stem cells are utilized for stimulation of perfusion of skeletal muscle. Said peripheral blood stem cells are characterized by ability to proliferate in vitro for a period of over 3 month and by expression of CD34, CXCR4, CD117, CD113, and c-met, and lack of differentiation associated markers, said markers may be selected from a group comprising of CD2, CD3, CD4, CD11, CD11a, Mac-1, CD14, CD16, CD19, CD24, CD33, CD36, CD38, CD45, CD56, CD64, CD68, CD86, CD66b, and HLA-DR.

In another aspect of the invention mesenchymal stem cells are utilized for stimulation of perfusion of skeletal muscle. Said mesenchymal stem cells express one or more of the following markers: STRO-1, CD105, CD54, CD106, HLA-I markers, vimentin, ASMA, collagen-1, fibronectin, LFA-3, ICAM-1, PECAM-1, P-selectin, L-selectin, CD49b/CD29, CD49c/CD29, CD49d/CD29, CD61, CD18, CD29, thrombomodulin, telomerase, CD10, CD13, STRO-2, VCAM-1, CD146, and THY-1, and do not express substantial levels of HLA-DR, CD117, and CD45. Said mesenchymal stem cells are derived from a group selected of: bone marrow, adipose tissue, endometrium, menstrual blood, umbilical cord blood, placental tissue, peripheral blood mononuclear cells, differentiated embryonic stem cells, and differentiated progenitor cells.

In another aspect of the invention germinal stem cells are utilized for stimulation of perfusion of skeletal muscle, said cells express markers selected from a group comprising of: Oct4, Nanog, Dppa5 Rbm, cyclin A2, Tex18, Stra8, Daz1, beta1- and alpha6-integrins, Vasa, Fragilis, Nobox, c-Kit, Sca-1 and Rex1.

In another aspect of the invention adipose tissue derived stem cells are utilized for stimulation of perfusion of skeletal muscle, wherein said adipose tissue derived stem cells express markers selected from a group comprising of: CD13, CD29, CD44, CD63, CD73, CD90, CD166, Aldehyde dehydrogenase (ALDH), and ABCG2, and said adipose tissue derived stem cells are a population of purified mononuclear cells extracted from adipose tissue capable of proliferating in culture for more than 1 month.

In another aspect of the invention exfoliated teeth derived stem cells are utilized for stimulation of perfusion of skeletal muscle, wherein said exfoliated teeth derived stem cells express markers selected from a group comprising of: STRO-1, CD 146 (MUC18), alkaline phosphatase, MEPE, and bFGF.

In another aspect of the invention hair follicle stem cells are utilized for stimulation of perfusion of skeletal muscle, wherein said cells express markers selected from a group comprising of: cytokeratin 15, Nanog, and Oct-4, and, wherein said hair follicle stem cells are capable of proliferating in culture for a period of at least one month, and wherein said hair follicle stem cells secrete one or more of the following proteins when grown in culture: basic fibroblast growth factor (bFGF), endothelin-1 (ET-1) and stem cell factor (SCF).

In another aspect of the invention dermal stem cells are utilized for stimulation of perfusion of skeletal muscle, wherein said cells express markers selected from a group comprising of: CD44, CD13, CD29, CD90, and CD105 and are capable of proliferating in culture for a period of at least one month

In another aspect of the invention parthenogenically derived stem cells are utilized for stimulation of perfusion of skeletal muscle, said parthenogenically derived stem cells may be generated by addition of a calcium flux inducing agent to activate an oocyte followed by enrichment of cells expressing markers selected from a group comprising of SSEA-4, TRA 1-60 and TRA 1-81.

In another aspect of the invention reprogrammed stem cells are utilized for stimulation of perfusion of skeletal muscle, said reprogrammed stem cells may be selected from a group comprising of: cells subsequent to a nuclear transfer, cells subsequent to a cytoplasmic transfer, cells treated with a DNA methyltransferase inhibitor, cells treated with a histone deacetylase inhibitor, cells treated with a GSK-3 inhibitor, cells induced to dedifferentiate by alteration of extracellular conditions, and cells treated with various combination of the mentioned treatment conditions. Said nuclear transfer comprises introducing nuclear material to a cell substantially enucleated, said nuclear material deriving from a host whose genetic profile is sought to be dedifferentiated. Said cytoplasmic transfer comprises introducing cytoplasm of a cell with a dedifferentiated phenotype into a cell with a differentiated phenotype, such that said cell with a differentiated phenotype substantially reverts to a dedifferentiated phenotype. Said DNA demethylating agent is selected from a group comprising of: 5-azacytidine, psammaplin A, and zebularine. Said histone deacetylase inhibitor is selected from a group comprising of: valproic acid, trichostatin-A, trapoxin A and depsipeptide.

In another aspect of the invention side population stem cells are utilized for stimulation of perfusion of skeletal muscle, said side population cells are identified based on expression multidrug resistance transport protein (ABCG2) or ability to efflux intracellular dyes such as rhodamine-123 and or Hoechst 33342. Side population cells may be derived from tissues such as pancreatic tissue, liver tissue, smooth muscle tissue, striated muscle tissue, cardiac muscle tissue, bone tissue, bone marrow tissue, bone spongy tissue, cartilage tissue, liver tissue, pancreas tissue, pancreatic ductal tissue, spleen tissue, thymus tissue, Peyer's patch tissue, lymph nodes tissue, thyroid tissue, epidermis tissue, dermis tissue, subcutaneous tissue, heart tissue, lung tissue, vascular tissue, endothelial tissue, blood cells, bladder tissue, kidney tissue, digestive tract tissue, esophagus tissue, stomach tissue, small intestine tissue, large intestine tissue, adipose tissue, uterus tissue, eye tissue, lung tissue, testicular tissue, ovarian tissue, prostate tissue, connective tissue, endocrine tissue, and mesentery tissue.

In another aspect of the invention committed progenitor cells are utilized for stimulation of perfusion of skeletal muscle, said committed progenitor cells are selected from a group comprising of: endothelial progenitor cells, neuronal progenitor cells, and hematopoietic progenitor cells. Said committed endothelial progenitor cells may be purified from the bone marrow. Said committed endothelial progenitor cells may be purified from peripheral blood. In one aspect, endothelial progenitors are collected from mobilized peripheral blood. Said mobilization may be accomplished by administration of a mobilizing agent or therapy. Said mobilizing agent may be selected from a group comprising of: G-CSF, M-CSF, GM-CSF, 5-FU, IL-1, IL-3, hyaluronic acid fragments, kit-L, VEGF, Flt-3 ligand, PDGF, EGF, FGF-1, FGF-2, TPO, IL-11, IGF-1, MGDF, NGF, HMG CoA)reductase inhibitors and small molecule antagonists of SDF-1. Said mobilization therapy may be selected from a group comprising of: exercise, hyperbaric oxygen, autohemotherapy by ex vivo ozonation of peripheral blood, and induction of SDF-1 secretion in an anatomical area outside of the bone marrow. In some aspects of the invention, committed endothelial progenitor cells express markers may be selected from a group comprising of: CD31, CD34, AC133, CD146 and flk1.

In one aspect of the invention committed hematopoietic cells are utilized for stimulation of perfusion of skeletal muscle, said committed hematopoietic cells may be purified from the bone marrow or peripheral blood. When purified from peripheral blood, committed hematopoietic progenitor cells are purified from peripheral blood of a patient whose committed hematopoietic progenitor cells are mobilized by administration of a mobilizing agent or therapy, said mobilizing agent may be selected from a group comprising of: G-CSF, M-CSF, GM-CSF, 5-FU, IL-1, IL-3, hyaluronic acid fragments, kit-L, VEGF, Flt-3 ligand, PDGF, EGF, FGF-1, FGF-2, TPO, IL-11, IGF-1, MGDF, NGF, HMG CoA)reductase inhibitors and small molecule antagonists of SDF-1. Said mobilization therapy may be selected from a group comprising of: exercise, hyperbaric oxygen, autohemotherapy by ex vivo ozonation of peripheral blood, and induction of SDF-1 secretion in an anatomical area outside of the bone marrow. In one aspect committed hematopoietic progenitor cells express the marker CD133 or CD34.

In one aspect of the invention cells described above as stem cells or committed progenitors may be administered systemically or in proximity to the pancreas in order to provide cellular and/or trophic support for regeneration of insulin producing cells.

In one aspect of the invention an anti-inflammatory agent may be administered to the patient receiving cells that increase skeletal muscle perfusion, and/or regeneration of insulin producing cells, said inflammatory inhibiting agents may inhibit molecular pathways such as the NF-kappa B pathway, the MyD88 pathway, the TNF signal transduction pathway, the Toll like receptor signal transduction pathway, and other pathways associated with upregulation of MHC expression, upregulation of C-reactive protein production, and upregulation of TNF alpha production. Antiinflammatory agents useful for practice of the invention are well known in the art and include Alclofenac; Alclometasone Dipropionate; Algestone Acetonide; Alpha Amylase; Alpha-lipoic acid; Alpha tocopherol; Amcinafal; Amcinafide; Amfenac Sodium; Amiprilose Hydrochloride; Anakinra; Anirolac; Anitrazafen; Apazone; Ascorbic Acid; Balsalazide Disodium; Bendazac; Benoxaprofen; Benzydamine Hydrochloride; Bromelains; Broperamole; Budesonide; Carprofen; Chlorogenic acid; Cicloprofen; Cintazone; Cliprofen; Clobetasol Propionate; Clobetasone Butyrate; Clopirac; Cloticasone Propionate; Cormethasone Acetate; Cortodoxone; Deflazacort; Desonide; Desoximetasone; Dexamethasone Dipropionate; Diclofenac Potassium; Diclofenac Sodium; Diflorasone Diacetate; Diflumidone Sodium; Diflunisal; Difluprednate; Diftalone; Dimethyl Sulfoxide; Drocinonide; Ellagic acid; Endrysone; Enlimomab; Enolicam Sodium; Epirizole; Etodolax; Etofenamate; Felbinac; Fenamole; Fenbufen; Fenclofenac; Fenclorac; Fendosal; Fenpipalone; Fentiazac; Flazalone; Fluazacort; Flufenamic Acid; Flumizole; Flunisolide Acetate; Flunixin; Flunixin Meglumine; Fluocortin Butyl; Fluorometholone Acetate; Fluquazone; Flurbiprofen; Fluretofen; Fluticasone Propionate; Furaprofen; Furobufen; Glutathione; Halcinonide; Halobetasol Propionate; Halopredone Acetate; Hesperedin; Ibufenac; Ibuprofen; Ibuprofen Aluminum; Ibuprofen Piconol; Ilonidap; Indomethacin; Indomethacin Sodium; Indoprofen; Indoxole; Intrazole; Isoflupredone Acetate; Isoxepac; Isoxicam; Ketoprofen; Lofemizole Hydrochloride; Lomoxicam; Loteprednol Etabonate; Lycopene; Meclofenamate Sodium; Meclofenamic Acid; Meclorisone Dibutyrate; Mefenamic Acid; Mesalamine; Meseclazone; Methylprednisolone Suleptanate; Morniflumate; Nabumetone; Naproxen; Naproxen Sodium; Naproxol; Nimazone; Oleuropein; Olsalazine Sodium; Orgotein; Orpanoxin; Oxaprozin; Oxyphenbutazone; Paranyline Hydrochloride; Pentosan Polysulfate Sodium; Phenbutazone Sodium Glycerate; Pirfenidone; Piroxicam; Piroxicam Cinnamate; Piroxicam Olamine; Pirprofen; Pycnogenol; Polyphenols; Prednazate; Prifelone; Prodolic Acid; Proquazone; Proxazole; Proxazole Citrate; Quercetin; Reseveratrol; Rimexolone; Romazarit; Rosmarinic acid; Rutin; Salcolex; Salnacedin; Salsalate; Sanguinarium Chloride; Seclazone; Sermetacin; Sudoxicam; Sulindac; Suprofen; Talmetacin; Talniflumate; Talosalate; Tebufelone; Tenidap; Tenidap Sodium; Tenoxicam; Tesicam; Tesimide; Tetrahydrocurcumin; Tetrydamine; Tiopinac; Tixocortol Pivalate; Tolmetin; Tolmetin Sodium; Triclonide; Triflumidate; Zidometacin; Zomepirac Sodium, IL-4, IL-10, IL-13, IL-20, IL-1 receptor antagonist, and TGF-beta.

### DETAILED DESCRIPTION OF THE ILLUSTRATED EMBODIMENTS

Embodiments of the present invention are described below. It is, however, expressly noted that the present invention is not limited to these embodiments, but rather the intention is that modifications that are apparent to the person skilled in the art and equivalents thereof are also included.

The current invention resides around the concept that insulin sensitivity can be increased through augmentation of muscular perfusion, and/or decreasing inflammation, and/or providing means for islet regeneration. Additionally, the invention provides means of treating a variety of conditions associated with insulin resistance outside of IDDM and NIDDM, conditions such as gestational diabetes, carbohydrate and lipid metabolism abnormalities, glucosuria, micro- and macrovascular disease, polyneuropathy and diabetic retinopathy, diabetic nephropathy, insulin resistance, impaired glucose tolerance (or glucose intolerance), obesity, hyperglycemia (elevated blood glucose concentration), hyperinsulinemia, hyperlipidemia, hyperlipoproteinemia, atherosclerosis, hypertension congenital or acquired digestion and absorption disorder including malabsorption syndrome; disease caused by loss of a mucosal barrier function of the gut; and protein-losing gastroenteropathy. Other conditions associated with above-normal blood glucose concentration either in an acute or chronic form are also embraced by the invention.

The invention teaches ways of utilizing cells, and in specific aspects, cells with ability to differentiate into other cells, for the purposes of stimulating muscle perfusion, decreasing inflammatory mediator production, and in some situations allowing for pancreatic regeneration. Also provided are means of inducing islet regeneration in an environment conducive to maintenance of viability and function of said islets or components thereof.

In one aspect of the invention, increasing angiogenic potential of a subject is performed with the purpose of increasing vascularity of the pancreas. The increase in angiogenic potential is performed through administration of angiogenic factors, cells with angiogenic ability, alone, or in combination. Angiogenesis is stimulated in the context of anti-inflammatory intervention, with, or without administration of cells capable of differentiating into insulin producing cells.

In one aspect of the invention a patient is treatment with agents known to stimulate generation of endogenous insulin producing cells, while at the same time increasing anti-inflammatory and angiogenic activity. Methods are known in the art for increasing endogenous insulin producing cell differentiation. One example of such a method is administration of combination EGF and gastrin, which has been demonstrated to induce insulin secretion through differentiation of endogenous stem cells into insulin producing cells (12-14).

In one aspect of the invention, anti-inflammatory agents are used together with stem cells capable of increasing angiogenesis, and/or inducing islet neogenesis.

In one embodiment of the invention, patients suffering from insulin resistance, having a state of NIDDM are treated by intramuscular administration of stem cells. It is known that 70-80% of post-prandial glucose is metabolized by skeletal muscle (15). In many patients with NIDDM, profound atherosclerotic deposits are known to inhibit circulation of the extremities. Without being bound to theory, inhibition of circulation may be occurring at vessels such as the femoral artery, the popliteal artery and/or the tibial arteries. Additionally inhibition of circulation may be occurring at the level of capillaries feeding various muscles. Impaired circulation is known to occur not only due to atherosclerosis, but also due to inhibited vasodilatory mechanisms (16). Due to inhibited circulation and vasodilatory responses, insulin activation of GLUT4 membrane localization and general insulin responsiveness is blunted. Accordingly in one embodiment of the current invention, the ability of muscles to respond to insulin is improved by administration of stem cells capable of restoring endothelial function, as well as inducing angiogenesis. Said stem cells useful for this purpose may be of autologous, endogenous, or allogeneic origin. In one particular embodiment a patient with NIDDM is treated with bone marrow cells administered intramuscularly. Bone marrow is collected from iliac crest by bone marrow aspiration. In one embodiment, approximately 200-500 ml of bone marrow is aspirated. In a more preferred embodiment 350 ml of bone marrow is aspirated. The aspirated bone marrow is placed into sterile plastic bags containing approximately 40-60 ml total of heparin sulfate (50 Units per bag) and Hank's buffered saline solution. The sterile plastic bags are contain a total of approximately 350 ml and 50 ml of bone marrow aspirate and heparin, respectively. The total heparinized bone marrow aspirate is the transferred into sterile bags for centrifugation and separation of mononuclear cells. In one embodiment, the heparinized bone marrow aspirate is transferred into eight 60 ml sterile GPS centrifuge containers designed specifically for the isolation and separation of a mononuclear and platelet rich suspension (GPS II Platelet Concentrate System, Biomet Biologics, Inc., Warsaw, Ind.). The transfer of the bone marrow suspension to the centrifuge containers is conducted on a sterile operating table utilizing 60 ml syringes. The centrifuge containers used are selected in this embodiment since they include a density-tuned dual buoy separation system and a central extraction tube. Alternative methods of collecting bone marrow and separating mononuclear cells are known in the art and the current technique is provided for illustrative purposes. Centrifugation is performed for approximately 15 minutes at approximately 3200 rpm in a centrifuge in sterile conditions. During centrifugation, the mononuclear cells and platelets migrate between the two buoys and collect within a fixed volume of fluid. The erythrocyte layer collects below the bottom buoy and the serum remains above the top buoy. Following centrifugation, the mononuclear cells and platelets are resuspended within the fixed volume of fluid, extracted with a 10 ml syringe via the central extraction tube, and transferred to the sterile field. Once cells are purified and placed in the sterile field, the cell suspension is separated into individual 0.5 ml aliquots using 1 ml tuberculin syringes. Patients with NIDDM are then treated by administration of said autologous bone marrow cells by injection that is approximately 1.5 cm deep into the gastrocnemius muscle. Injections may be performed to deliver a total number of bone marrow cells ranging from 10 million to 10 billion mononuclear cells. In a preferred embodiment injections of approximately 1-3 billion mononuclear cells are administered. Said injections may be performed with a total injection volume of 10-50 ml, with injections being distributed on a grid placed on the gastrocnemius muscle. Number of injections may range from 1-100 injections, with an optimum number ranging approximately from 10-50 injections, and more optimally between 20-30 injections. Injection of bone marrow mononuclear cells may be performed specifically in an area of occlusion identified by methods known in the art, such as digital subtractive angiography, Doppler imaging, positron emission tomography, and ultrasound. Alternatively, administration of bone marrow cells may be performed in areas in which occlusion is suspected by not established. Additionally, means of assessing tissue oxygenation such as transcutaneous pulse oximetry may be used to identify muscular areas deficient in oxygenation. Deficiencies in general circulation may also be identified by measurements such as toe pulse, or by the ankle-brachial index. In one embodiment administration of bone marrow stem cells is performed in the gastrocnemious muscle is the ankle brachial index is below 0.9. In other embodiments, administration of bone marrow stem cells is performed in various muscles regardless of perfusion status. For example, patients with NIDDM may be injected with numerous aliquots of bone marrow in major skeletal muscles. Examples of major skeletal muscles suitable for injection include: the deltoid, pectoralis major, biceps, rectus abdominus, external oblique, gluteus medius, gluteus maximus, soleus, tibialis anterior, vastus medialis, vastus intermedius, vastus lateralis, rectus femoris, and the sartorious muscles.

The effects of intramuscular bone marrow mononuclear cell administration may be observed not only by ability to increase perfusion, but also ability to augment the flow mediated dilation response. Most optimally, the effect of cell administration is assessed by various means known in the art for quantification of insulin sensitivity. For example, the hyperinsulinemic-euglycemic clamp technique considered a golden standard for this purpose, however due to impracticalities such as time and expense, other techniques may also be used. Said techniques include the frequently sampled IV glucose tolerance test (FSIVGTT), insulin tolerance test (ITT), insulin sensitivity test (IST), the continuous infusion of glucose with model assessment (CIGMA) and the oral glucose tolerance test (OGTT).

Treatment with bone marrow mononuclear cells may be performed, in some embodiments of the invention, in conjunction with cytokines known to mobilize endogenous stem cells. It is known that intramuscular administration of bone marrow mononuclear cells causes systemic mobilization of endogenous CD34 stem cells from the bone marrow (17). Accordingly, the current invention teaches that subsequent to administration of bone marrow mononuclear cells into muscle of a patient with NIDDM, augmentation of endogenous stem cell mobilization will evoke an enhanced therapeutic effect. Since the intramuscularly administered stem cells possess chemotactic activity, the mobilization of bone marrow stem cells through administration of factors such as G-CSF will augment therapeutic effect. Administration of G-CSF may be performed concurrently with intramuscular injection of bone marrow cells, or may be performed near the timepoint associated with maximal mobilization of CD34 cells. Said timepoint may be determined experimentally, or may be based on previously published data. It is reported, for example, that maximal CD34 mobilization subsequent to administration of bone marrow cells intramuscularly occurs around day 30. Accordingly, in one embodiment of the invention, G-CSF is administered prior to day 30, at concentrations sufficient to evoke endogenous CD34 mobilization. In one embodiment, G-CSF is administered at a concentration of approximately 60 migrograms per day be subcutaneous injection for 5 days. Administration may be performed, for example, starting on day 25 subsequent to intramuscular injection of bone marrow cells. In some embodiments, it is important to concurrently administer heparin so as to avoid the possibility of causing embolism due to high systemic leukocyte counts caused by the G-CSF injection. This is particularly important in patients with NIDDM who are already at a higher risk of embolisms in comparison to the general population. Anticoagulation methods are well known in the art and may utilize agents besides heparin. However, if heparin anticoagulation is used, then approximate doses of 10,000 units per day may be useful.

In another embodiment stem cells, such as bone marrow mononuclear cells are administered as described above in combination with agents known to increase stem cell activity. Such agents may include, for example, erythropoietin (18), prolactin (19), human chorionic gonadotropin (as described in U.S. Pat. No. 5,968,513 and incorporated by reference), gastrin (20), EGF (12), FGF (21), and/or VEGF (22). In some situations administration of inhibitors of inhibitors of stem cells is also provided in the invention along with the use of exogenous stem cell administration. For example, administration of neutralizers of TNF alpha are concurrently administered with stem cells to derepress inhibitory effects of this cytokine on circulating stem cells, as previously reported in rheumatoid arthritis (23).

In another embodiment of the invention, stem cells of other origin may be used. Said stem cells are endowed with angiogenic potential through culture with various angiogenic agents. Said agents are well known in the art and include cytokines such as EGF, VEGF, FGF, EGF, and angiopoietin.

In one embodiment of the invention, stem cells are collected from amniotic fluid or amniotic membrane. Said amniotic derived mononuclear cells may be utilized therapeutically in an unpurified manner subsequent to matching. Said amniotic stem cells are administered locally, intramuscularly or systemically in a patient suffering from insulin resistance. In other embodiments amniotic stem cells are substantially purified based on expression of markers such as SSEA-3, SSEA4, Tra-1-60, Tra-1-81 and Tra-2-54, and subsequently administered. In other embodiments cells are cultured, as described in U.S. patent application # 20050054093, expanded, and subsequently infused into the patient. Amniotic stem cells are described in the following references (24-26). One particular aspect of amniotic stem cells that makes them amenable for use in practicing certain aspects of the current invention is their bi-phenotypic profile as being both mesenchymal and endothelial progenitors this allows for anti-inflammatory, as well as angiogenic function (25, 27). This property is useful for treatment of patients with insulin resistance and associated diseases that would benefit from angiogenesis, but also from the anti-inflammatory effects of mesenchymal stem cells. The use of amniotic fluid stem cells is particularly useful in situations such as ischemia associated pathologies and/or inflammatory states, in which hypoxia is known to perpetuate degenerative processes. The various embodiments of the invention for other stem cells described in this disclosure can also be applied for amniotic fluid stem cells. In some embodiments, said amniotic stem cells may be administered with a population of matched tolerogenic cells into the allogeneic recipient so as not to be rejected by said recipient.

In another embodiment, allogeneic, or autologous donors that have been matched with HLA or mixed lymphocyte reaction are mobilized by administration of G-CSF (filgrastim: neupogen) at a concentration of approximately 10 ug/kg/day by subcutaneous injection for 2-7 days, more preferably 4-5 days. Peripheral blood mononuclear cells are collected using an apheresis device such as the AS104 cell separator (Fresenius Medical). 1-40×10⁹ mononuclear cells are collected, concentrated and administered locally, injected systemically, or in an area proximal to the region pathology associated with the given degenerative disease. In situations where ischemia is localized cellular administration may be performed within the context of the current invention. Methods of identification of such areas of ischemia is routinely known in the art and includes the use of techniques such as nuclear or MRI imagining. Variations of this procedure may include steps such as subsequent culture of cells to enrich for various populations known to possess angiogenic and/or anti-inflammatory, and/or anti-remodeling, and/or regenerative properties. Additionally cells may be purified for specific subtypes before and/or after culture. Treatments can be made to the cells during culture or at specific timepoints during ex vivo culture but before infusion in order to generate and/or expand specific subtypes and/or functional properties. The various embodiments of the invention for other stem cells described in this disclosure can also be applied for circulating peripheral blood stem cells.

In another embodiment of the invention, allogeneic or autologous adipose tissue derived stem cells are used as a stem cell source. Said adipose tissue derived stem cells express markers such as CD9; CD29 (integrin beta 1); CD44 (hyaluronate receptor); CD49d,e (integrin alpha 4, 5); CD55 (decay accelerating factor); CD105 (endoglin); CD106 (VCAM-1); CD166 (ALCAM). These markers are useful not only for identification but may be used as a means of positive selection, before and/or after culture in order to increase purity of the desired cell population. In terms of purification and isolation, devices are known to those skilled in the art for rapid extraction and purification of cells adipose tissues. U.S. Pat. No. 6,316,247 describes a device which purifies mononuclear adipose derived stem cells in an enclosed environment without the need for setting up a GMP/GTP cell processing laboratory so that patients may be treated in a wide variety of settings. One embodiment of the invention involves attaining 10-200 ml of raw lipoaspirate, washing said lipoaspirate in phosphate buffered saline, digesting said lipoaspirate with 0.075% collagenase type I for 30-60 min at 37° C. with gentle agitation, neutralizing said collagenase with DMEM or other medium containing autologous serum, preferably at a concentration of 10% v/v, centrifuging the treated lipoaspirate at approximately 700-2000 g for 5-15 minutes, followed by resuspension of said cells in an appropriate medium such as DMEM. Cells are subsequently filtered using a cell strainer, for example a 100 μm nylon cell strainer in order to remove debris. Filtered cells are subsequently centrifuged again at approximately 700-2000 g for 5-15 minutes and resuspended at a concentration of approximately 1×10⁶/cm² into culture flasks or similar vessels. After 10-20 hours of culture non-adherent cells are removed by washing with PBS and remaining cells are cultured at similar conditions as described for culture of cord blood derived mesenchymal stem cells. Upon reaching a concentration desired for clinical use, cells are harvested, assessed for purity and administered in a patient in need thereof as described above. The various embodiments of the invention for other stem cells described in this disclosure can also be applied for adipose derived stem cells.

In one embodiment of the invention, allogeneic or autologous pluripotent stem cells derived from deciduous teeth (baby teeth) are used. Said stem cells have been recently identified as a source of stem cells with ability to differentiate into endothelial, neural, and bone structures. Said pluripotent stem cells have been termed “stem cells from human exfoliated deciduous teeth” (SHED). One of the embodiments of the current invention involves utilization of this novel source of stem cells for the treatment of various degenerative conditions without need for immune suppression. In one embodiment of the invention, SHED cells are administered systemically or locally into a patient with a degenerative condition at a concentration and frequency sufficient for induction of therapeutic effect. SHED cells can be purified and used directly, certain sub-populations may be concentrated, or cells may be expanded ex vivo under distinct culture conditions in order to generate phenotypes desired for maximum therapeutic effect. Growth and expansion of SHED has been previously described by others. In one particular method, exfoliated human deciduous teeth are collected from 7- to 8-year-old children, with the pulp extracted and digested with a digestive enzyme such as collagenase type I. Concentrations necessary for digestion are known and may be, for example 1-5 mg/ml, or preferable around 3 mg/ml. Additionally dispase may also be used alone or in combination, concentrations of dispase may be 1-10 mg/ml, preferably around 4 mg/ml. Said digestion is allowed to occur for approximately 1 h at 37° C. Cells are subsequently washed and may be used directly, purified, or expanded in tissue culture. Recently, the commercial business of tooth stem cell banking has been announced at the website http://www.bioeden.com. The various embodiments of the invention for other stem cells described in this disclosure can also be applied for for exfoliated teeth stem cells.

One embodiment of the current invention is the use of allogeneic or hair follicle derived stem cells for treatment of insulin resistance and associated conditions. Said cells may be used therapeutically once freshly isolated, or may be purified for particular sub-populations, or may be expanded ex vivo prior to use. Purification of hair follicle stem cells may be performed from cadavers, from healthy volunteers, or from patients undergoing plastic surgery. Upon extraction, scalp specimens are rinsed in a wash solution such as phosphate buffered saline or Hanks and cut into sections 0.2-0.8 cm. Subcutaneous tissue is de-aggregated into a single cell suspension by use of enzymes such as dispase and/or collagenase. In one variant, scalp samples are incubated with 0.5% dispase for a period of 15 hours. Subsequently, the dermal sheath is further enzymatically de-aggregated with enzymes such as collagenase D. Digestion of the stalk of the dermal papilla, the source of stem cells is confirmed by visual microscopy. Single cell suspensions are then treated with media containing fetal calf serum, and concentrated by pelletting using centrifugation. Cells may be further purified for expression of markers such as CD34, which are associated with enhanced proliferative ability. In one embodiment of the invention, collected hair follicle stem cells are induced to differentiate in vitro into neural-like cells through culture in media containing factors such as FGF-1, FGF-2, NGF, neurotrophin-2, and/or BDNF. Confirmation of neural differentiation may be performed by assessment of markers such as Muhashi, polysialyated N-CAM, N-CAM, A2B5, nestin, vimentin glutamate, synaptophysin, glutamic acid decarboxylase, serotonin, tyrosine hydroxylase, and GABA. Said neuronal cells may be administered systemically, intramuscularly or locally in a patient with insulin resistance or associated diseases. Differentiation towards other phenotypes may also be performed within the context of the current invention. The various embodiments of the invention for other stem cells described in this disclosure can also be applied for hair follicle stem cells.

In one embodiment of the invention, very early, immature stem cells are used in an allogeneic or autologous manner. Said stem cells being parthenogenically derived stem cells that can be generated by addition of a calcium flux inducing agent to activate oocytes, followed by purifying and expanding cells expressing embryonic stem cell markers such as SSEA-4, TRA 1-60 and/or TRA 1-81. Said parthenogenically derived stem cells are totipotent and can be used in a manner similar to that described other stem cells in the practice of the current invention. One specific methodology for generation of parthenogenically derived stem cells involves maturing oocytes by culture 36 hour in CMRL-1066 media supplemented with 20% FCS, 10 units/ml pregnant mare serum, 10 units /ml HCG, 0.05 mg/ml penicillin, and 0.075 mg/ml streptomycin. Mature metaphase II eggs are subsequently activated with calcium flux by incubation with 10 uM ionomycin for 8 minutes, followed by culture with 2 mM 6-dimethylaminopurine for 4 hours. The inner cell mass is subsequently isolated by immunosurgical technique and cells are cultured on a feeder layer in a manner similar to culture of embryonic stem cells (28). The various embodiments of the invention for other stem cells described in this disclosure can also be applied for parthenogenically derived stem cells.

Unique, tissue-specific stem cells may also be used in the autologous or allogeneic setting for the practice of the current invention. These cells may be used whole, or induced to differentiate into endothelial or endothelial precursor cells. Cells expressing the ability to efflux certain dyes, including but not limited to rhodamin-123 are associated with stem cell-like properties (29). Said cells can be purified from tissue subsequent to cell dissociation, based on efflux properties. Accordingly, in one embodiment of the current invention, tissue derived side population cells may be utilized either freshly isolated, sorted into subpopulations, or subsequent to ex vivo culture, for the treatment of degenerative conditions. For use in the invention, side population cells may be derived from tissues such as pancreatic tissue, liver tissue, smooth muscle tissue, striated muscle tissue, cardiac muscle tissue, bone tissue, bone marrow tissue, bone spongy tissue, cartilage tissue, liver tissue, pancreas tissue, pancreatic ductal tissue, spleen tissue, thymus tissue, Peyer's patch tissue, lymph nodes tissue, thyroid tissue, epidermis tissue, dermis tissue, subcutaneous tissue, heart tissue, lung tissue, vascular tissue, endothelial tissue, blood cells, bladder tissue, kidney tissue, digestive tract tissue, esophagus tissue, stomach tissue, small intestine tissue, large intestine tissue, adipose tissue, uterus tissue, eye tissue, lung tissue, testicular tissue, ovarian tissue, prostate tissue, connective tissue, endocrine tissue, and mesentery tissue. Purification of side population cells can be performed, in one embodiment, by resuspending dissociated cardiac valve cells at 10⁶ cells/ml, and staining with 6.0 μg/ml of Hoechst 33342 in calcium- and magnesium-free HBSS+ (supplemented with 2% FCS, 10 mM Hepes, and 1% penicillin/streptomycin) medium for 90 min at 37° C. Cells are then run on a flow cytometer and assessed for efflux of Hoechst 33342. Purified cells may be assessed for ability to form cardiac spheres, this may be performed by suspending said side population cells at a density of 1-2×106 cells/ml in 10-cm uncoated dishes in DME/M199 (1:1) serum-free growth medium containing insulin (25 μg/ml), transferin (100 μg/ml), progesterone (20 nM), sodium selenate (30 nM), putrescine (60 nM), recombinant murine EGF (20 ng/ml), and recombinant human FGF2. Half of the medium is changed every 3 d. Passaging may be performed using 0.05% trypsin and 0.53 mM EDTA-4Na every 7-14 d. Cardiospheres are then dissociated into a single-cell suspension then used either for therapeutic purposes, or for evaluating therapeutic ability in vitro or in animal models before clinical use. Said cardiospheres can be induced to differentiate into endothelial cells by culture in angiogenic factors prior to administration. These methods have been described for side population stem cells of other tissues in publications to which the practitioner of the invention is referred to (30-32). The various embodiments of the invention for other stem cells described in this disclosure can also be applied for side population stem cells.

In another embodiment of the invention, “young” stem cells are used to compensate for deteriorating function of senescent tissue. The term “young” is used to denote cells derived from a donor of an age younger than the recipient. In some embodiments, young cells may be cells of the same recipient that were collected at an earlier date to infusion of cells. There are certain advantages for utilization of young cells for the practice of the current invention. For example, it is known that aged animals possess impaired physiological responses in comparison to younger animals. Aging is known to be associated with impaired insulin responsiveness (33, 34). In some cases senescence is associated with increased production of inflammatory cytokines such as TNF-alpha, which cause insulin resistance. For example, it was demonstrated that antibodies to TNF-alpha are capable of inhibiting age-related insulin resistance of muscles of Sprague-Dawley rats (35). The ability of stem cells to differentiate into a wide variety of muscles is known both from cord blood derived sources (36-38), as well as from bone marrow (39-54) and adipose sources (55-58). Additionally, it is known that stem cells from a young donor can integrate into tissue of an older recipient and contribute to biological functions. For example, in an experiment by Edelberg's group, it was demonstrated that 3 month old ROSA beta galactosidase transgenic bone marrow cells, when transferred into an 18-month old recipient are capable of entering the bone marrow and causing chimeric hematopoiesis in absence of recipient conditioning (59). More interestingly, it was demonstrated that endothelial progenitor cells from the young 3 month old bone marrow donor are capable of “rejuvenating” 18 month old recipient mouse ability to sustain vascularization of neonatal hearts transplanted ectopically. Specifically, when 18 month old recipients were transplanted with neonatal hearts, donor hearts lost viability due to lack of vascularization. If 18 month old bone marrow cells were administered into the 18 month old recipient, ability to vascularize the neonatal heart was still impaired. However, 3 month old bone marrow infusion was capable of establishing vascularization in a dose-dependent and PDGF-B dependent manner.

In one embodiment of the invention, stem cells, substantially younger than a recipient are administered into said recipient for production of cells that directly or indirectly increase responsiveness to insulin. As previously stated, stem cells derived from cord blood, bone marrow, and adipose tissue are capable of differentiating into skeletal muscle. Taking the observation that younger cells are capable of integrating with older tissue and re-establishing function of older tissue, the invention teachings the use of younger stem cells for increasing responsiveness to insulin. In one embodiment cord blood stem cells are utilized as a source of “young” stem cells for generation of cells similar to skeletal muscle cells in vivo in order to decrease insulin resistance. This is not to be interpreted as being bound to theory since the differentiation into muscle-like cells is one of several mechanisms by which the invention discloses ability of cord blood to reverse insulin resistance. In one embodiment, said cord blood stem cells are obtain from a cord blood sample obtained from a healthy pregnancy. Umbilical cord blood is purified according to routine methods (60). In one embodiment, a 16-gauge needle from a standard Baxter 450-ml blood donor set containing CPD A anticoagulant (citrate/phosphate/dextrose/adenine) (Baxter Health Care, Deerfield, Ill.) is inserted and used to puncture the umbilical vein of a placenta obtained from a mother tested for viral and bacterial infections according to international donor standards. Cord blood is allowed to drain by gravity so as to drip into the blood bag. The placenta is placed in a plastic-lined, absorbent cotton pad suspended from a specially constructed support frame in order to allow collection and reduce the contamination with maternal blood and other secretions. The 63 ml of CPD A used in the standard blood transfusion bag, calculated for 450 ml of blood, is reduced to 23 ml by draining 40 ml into a graduated cylinder just prior to collection. An aliquot of the cord blood is removed for safety testing according to the standards of the National Marrow Donor Program (NMDP) guidelines. Safety testing includes routine laboratory detection of human immunodeficiency virus 1 and 2, human T-cell lymphotropic virus I and II, Hepatitis B virus, Hepatitis C virus, Cytomegalovirus and Syphilis. Subsequently, 6% (wt/vol) hydroxyethyl starch is added to the anticoagulated cord blood to a final concentration of 1.2%. The leukocyte rich supernatant is then separated by centrifuging the cord blood hydroxyethyl starch mixture in the original collection blood bag (50×g for 5 min at 10° C.). The leukocyte-rich supernatant is transferred from the bag into a 150-ml Plasma Transfer bag (Baxter Health Care) and centrifuged (400× g for 10 min) to sediment the cells. Surplus supernatant plasma is transferred into a second plasma transfer bag without severing the connecting tube. Finally, the sedimented leukocytes are resuspended in supernatant plasma to a total volume of 20 ml. Approximately 5×10⁸ -7'10⁹ nucleated cells are obtained per cord. Cells are cryopreserved according to the method described by Rubinstein et al (60).

In some situations, matching of donor cells to recipient is performed, in other situations it is not. For example, a group of 25 cord blood stem cell sources, purified and cryopreserved as described above, is available for treatment of a patient in need of stem cell therapy. An aliquot of mononuclear cells from each of said 25 cord blood stem cell source is taken, said aliquot comprising approximately 10⁵ cells. Said cells are plated in Nunc 96-well plates at a concentration of 10⁴ cells per well in 9 wells in a volume of 100 uL per well. Prior to plating, said cells are washed and reconstituted in DMEM-LG media (Life Technologies), supplemented with 10% heat-inactivated fetal calf serum. Said cord blood cells are considered “stimulators” for the purpose of the matching procedure. In order to generate “responder” cells, 20 ml of peripheral blood is extracted from the patient in need of stem cell therapy through venipuncture. Said 20 ml of peripheral blood is heparinized by drawing in a heparinized Vacutainer TM, is layered on Ficoll™ density gradient and centrifuged for approximately 60 minutes at 500 g. The mononuclear layer is harvested and washed in phosphate buffered saline supplemented with 3% fetal calf serum. For every 9 wells of stimulator cells, to 3 wells, a concentration of 10⁴ responder cells are added, to 3 wells a concentration of 10⁵ responder cells are added, and to 3 wells, media with no cells are added in order to have a control for spontaneous activity of stimulator cells. Responder cells are reconstituted in DMEM-LG media, supplemented with 10% heat-inactivated fetal calf serum before being added to stimulator cells. Responder cells and media comprise a volume of 100 uL before being added to stimulator cells. Additionally, in order to have a control for spontaneous activity of responder cells, 10⁴ and 10⁵ responder cells in a volume of 100 uL are added in triplicate to 100 uL of media without stimulator cells. To have a control for background or other contaminations, 3 wells are plated with 200 uL of media alone. Accordingly, the total culture consists of 25 stem cell sources×9 wells=225 wells, that is, a total of three 96-well plates are used. Additionally, 9 wells are used for the responder controls in which no stimulator cells, or no cells at all are added. Seventy-two-hour mixed lymphocyte reaction is performed and the cells were pulsed with 1 μCi [3H]thymidine for the last 18 h. The cultures are harvested onto glass fiber filters (Wallac, Turku, Finland). Radioactivity is counted using a Wallac 1450 Microbeta liquid scintillation counter and the data were analyzed with UltraTerm 3 software (Microsoft, Seattle, Wash.). If lymphocyte proliferation is more than 2 fold higher as compared to lymphocytes cultured without stimulator cells, when subtracting the background proliferation of stimulators alone, then the cord blood batch is not used for therapy. According to this criteria, 2 of the 25 batches of stem cell sources may be chosen for administration into said patient. Cells purified may be utilized for intramuscular injection.

In another embodiment cord blood is used with or without matching to the recipient, however steps are take so as to deplete the cord blood of specific immunogenic components that may cause host versus graft, and/or alternatively, the graft is manipulated so as to neutralize immunological cells that may have the potential to cause graft versus host. Specifically, cord blood mononuclear cells are concentrated in Good Manufacturing Practices (GMP) grade-Hanks balanced salt solution (containing Ca2+). Cells are washed previously to concentration so that said cells are substantially free from plasma and depleted of red blood cells and granulocytes. The volume of the mononuclear cell suspension is adjusted so that the cell density is approximately 3×10⁷/mL, and CAMPATH-1M or CAMPATH-1H is added to give a final concentration of 0.1 mg/mL. The mixture is incubated for 15 minutes at room temperature, and then recipient serum is added to achieve final concentration of 25% (vol/vol). The mixture is subsequently incubated for a further 30 minutes at 37° C. The treated cord blood cells are washed once, assessed for viability, and infused into a patient in need of therapy.

The ability of stem cells to differentiate into various tissues is well known, however, a lesser known ability of various stem cells is their anti-inflammatory function. It is established that NIDDM is associated with elevation of inflammatory mediators. This was elegantly overviewed in a review by Pickup et al who described a “low grade inflammation” as part of the process associated with development of insulin resistance and subsequent NIDDM. This is based on observations that elevated circulating inflammatory markers such as C-reactive protein and interleukin-6 predict the development of type 2 diabetes, and several drugs with anti-inflammatory properties lower both acute-phase reactants and glycemia (aspirin and thiazolidinediones) and possibly decrease the risk of developing type 2 diabetes (statins). Additionally Pickup postulates that features of type 2 diabetes, such as fatigue, sleep disturbance, and depression may be the result of systemic “hypercytokinemia” (61). It is known that TNF-alpha and IL-6 are secreted at a basal level by the adipose compartment and correlations have been made between systemic levels of these cytokines and resistance to insulin. For example, Kern et al measured TNF and IL-6 levels in non-diabetic lean and obese patients. When lean [body mass index (BMI) <25 kg/m(2)] and obese (BMI 30-40 kg/m(2)) subjects were compared, there was a 7.5-fold increase in TNF secretion, and the TNF secretion was inversely related to insulin sensitivity as measured by the intravenous glucose tolerance test (62). Numerous other studies have demonstrated high levels of TNF in plasma of patients that are insulin resistant (63, 64). Additionally, reduction in TNF-alpha is associated with response to various insulin sensitizers (65). The ability of TNF-alpha to induce insulin resistance is believed to be based on induction of serine phosphorylation of insulin receptor substrate-1 (IRS-1). IRS-1 serine phosphorylation causes dissociation of IRS proteins from the insulin receptor, thus blocking insulin signal transduction (66). Despite the important role of TNF-alpha in insulin resistance, it is not the only causative factor. Treatment with TNF-alpha blocking agents appears not to increase insulin sensitivity (67, 68). This, however, is most likely due to the plethora of inflammatory agents such as leptin, IL-6, resistin, visfatin and IL-1 that are secreted by adipose tissue and associated with insulin resistance in addition to TNF-alpha (69, 70). In rheumatoid arthritis TNF-alpha is one of the major cytokines produced, and as a result insulin resistance develops. Interestingly, blockade of TNF-alpha using infliximab in RA patients results in increased insulin sensitivity (71). This finding may be explained by the fact that RA is associated with one major inflammatory mediator, whereas obesity is associated with several. Accordingly, in one embodiment of the invention, stem cells are used to induce an anti-inflammatory state or to reduce inflammation in a patient with NIDDM. Said inflammatory state may be diagnosed by many means available to one of skill in the art, including assessment of C-reactive protein levels, IL-1, IL-6, TNF, leptin, and IL-18. Various stem cell sources may be used in the practice of the invention. Additionally, the combination of stem cell for the generation of angiogenesis, together with stem cells for the induction of an anti-inflammatory state is disclosed in the current invention. The cells that are useful may include, in some embodiments, mesenchymal stem cells. These cells have been shown to possess immune suppressive and anti-inflammatory functions. For example, it was demonstrated in a murine model that flk-1+Sca-1-marrow derived mesenchymal stem cell transplantation leads to permanent donor-specific immunotolerance in allogeneic hosts and results in long-term allogeneic skin graft acceptance (72). These studies have demonstrated that inhibition of both inflammatory cytokine production, as well as blocking of donor-reactive T cell proliferation was achieved. Other studies have shown that mesenchymal stem cells are inherently immunosuppressive through production of PGE-2, interleukin-10 and expression of the tryptophan catabolizing enzyme indoleamine 2,3,-dioxygenase as well as galectin-1 (73, 74). These stem cells also have the ability to non-specifically modulate the immune response through the suppression of dendritic cell maturation and antigen presenting abilities (75, 76). By inhibiting dendritic cell functions, it is within the scope of the patent to teach that mesenchymal stem cells may reduce non-T cell inflammatory signals. This includes inhibition of macrophage inflammatory activity which has been reported to be critical in NIDDM (77). In some embodiments, treatment of NIDDM is performed by introduction of freshly isolated mesenchymal stem cells, or populations of cells containing mesenchymal stem cells into the patient. Immune suppressive activity of mesenchymal stem cells is not dependent on prolonged culture of mesenchymal stem cells since it was demonstrated by others that functional induction of allogeneic T cell apoptosis occurs using freshly isolated, irradiated, mesenchymal stem cells (78). It has also been shown that mesenchymal stem cells have the ability to preferentially induce expansion of antigen specific T regulatory cells with the CD4+ CD25+ phenotype (79). Mesenchymal cells can antigen specifically inhibit immune responses as observed in a murine model of multiple sclerosis, experimental autoimmune encephalomyelitis, in which administration of these cells lead to inhibition of disease onset (80).

In some embodiments of the invention, stem cell populations are used together with agents known to stimulate production of insulin or protect islets from damage. For example, such agents may be amylin analogs. These compounds duplicate the effect of amylin by delaying gastric emptying, decreasing postprandial glucagon release, and modulating appetite. Pramlintide acetate, sold under the name Symlin is indicated as an adjunct to mealtime insulin for the treatment of patients with type 1 and type 2 diabetes. In numerous clinical trials, adjunctive pramlintide treatment resulted in improved postprandial glucose control and significantly reduced A1C and body weight compared with insulin alone. Numerous patents have been issued for various agents capable of stimulating insulin secretion and/or sensitizing peripheral tissue to insulin activity. These include, for example, U.S. Pat. Nos. 6,121,282, 6,057,343, 6,048,842, 6,037,359, 6,030,990, 5,990,139, 5,981,510, 5,980,902, 5,955,481, 5,929,055, 5,925,656, 5,925,647, 5,916,555, 5,900,240, 5,885,980, 5,849,989, 5,837,255, 5,830,873, 5,830,434, 5,817,634, 5,783,556, 5,756,513, 5,753,790, 5,747,527, 5,731,292, 5,728,720, 5,708,012, 5,691,386, 5,681,958, 5,677,342, 5,674,900, 5,545,672, 5,532,256, 5,531,991, 5,510,360, 5,480,896, 5,468,762, 5,444,086, 5,424,406, 5,420,146, RE34,878, 5,294,708, 5,268,373, 5,258,382, 5,019,580, 4,968,707, 4,845,231, 4,845,094, 4,816,484, 4,812,471, 4,740,521, 4,716,163, 4,695,634, 4,681,898, 4,622,406, 4,499,279, 4,467,681, 4,448,971, 4,430,337, 4,421,752, 4,419,353, 4,405,625, 4,374,148, 4,336,391, 4,336,379, 4,305,955, 4,262,018, 4,220,650, 4,207,330, 4,195,094, 4,172,835, 4,164,573, 4,163,745, 4,141,898, 4,129,567, 4,093,616, 4,073,910, 4,052,507, 4,044,015, 4,042,583, 4,008,245, 3,992,388, 3,987,172, 3,961,065, 3,954,784, 3,950,518, 3,933,830, which are incorporated herein by reference.

### EXAMPLE

### Example 1

### Increased Insulin Responsiveness in Type 2 Diabetes

A group of 100 patients are recruited with type 2 diabetes receiving daily insulin injections. 50 patients are treated with placebo control and 50 receive allogeneic cord blood derived CD34 cells. Cells are injected intramuscularly in the gastrocnemius muscle as described in the literature (Durdu et al. J Vasc Surg. 2006 October;44(4):732-9) with a concentration of 40 million cells per limb. Cord blood CD34 extraction and expansion are described below. Umbilical cord blood is purified according to routine methods ((Rubinstein, et al. Processing and cryopreservation of placental/umbilical cord blood for unrelated bone marrow reconstitution. Proc Natl Acad Sci USA 92:10119-10122). Briefly, a 16-gauge needle from a standard Baxter 450-ml blood donor set containing CPD A anticoagulant (citrate/phosphate/dextrose/adenine) (Baxter Health Care, Deerfield, Ill.) is inserted and used to puncture the umbilical vein of a placenta obtained from healthy delivery from a mother tested for viral and bacterial infections according to international donor standards. Cord blood is allowed to drain by gravity so as to drip into the blood bag. The placenta is placed in a plastic-lined, absorbent cotton pad suspended from a specially constructed support frame in order to allow collection and reduce the contamination with maternal blood and other secretions. The 63 ml of CPD A used in the standard blood transfusion bag, calculated for 450 ml of blood, is reduced to 23 ml by draining 40 ml into a graduated cylinder just prior to collection. This volume of anticoagulant matches better the cord volumes usually retrieved (<170 ml).

An aliquot of the blood is removed for safety testing according to the standards of the National Marrow Donor Program (NMDP) guidelines. Safety testing includes routine laboratory detection of human immunodeficiency virus 1 and 2, human T-cell lymphotropic virus I and II, Hepatitis B virus, Hepatitis C virus, Cytomegalovirus and Syphilis. Subsequently, 6% (wt/vol) hydroxyethyl starch is added to the anticoagulated cord blood to a final concentration of 1.2%. The leukocyte rich supernatant is then separated by centrifuging the cord blood hydroxyethyl starch mixture in the original collection blood bag (50×g for 5 min at 10° C.). The leukocyte-rich supernatant is expressed from the bag into a 150-ml Plasma Transfer bag (Baxter Health Care) and centrifuged (400×g for 10 min) to sediment the cells. Surplus supernatant plasma is transferred into a second plasma Transfer bag without severing the connecting tube. Finally, the sedimented leukocytes are resuspended in supernatant plasma to a total volume of 20 ml. Approximately 5×10⁸ -7×10⁹ nucleated cells are obtained per cord. Cells are cryopreserved according to the method described by Rubinstein et al (Rubinstein, et al. Processing and cryopreservation of placental/umbilical cord blood for unrelated bone marrow reconstitution. Proc Natl Acad Sci USA 92:10119-10122). for subsequent cellular therapy. CD34 cells are expanded by culture. CD34+ cells are purified from the mononuclear cell fraction by immuno-magnetic separation using the Magnetic Activated Cell Sorting (MACS) CD34+Progenitor Cell Isolation Kit (Miltenyi-Biotec, Auburn, Calif.) according to manufacturer's recommendations. The purity of the CD34+cells obtained ranges between 95% and 98%, based on Flow Cytometry evaluation (FACScan flow cytometer, Becton-Dickinson, Immunofluorometry systems, Mountain View, Calif.). Cells are plated at a concentration of 10.sup.4 cells/ml in a final volume of 0.5 ml in 24 well culture plates (Falcon; Becton Dickinson Biosciences) in DMEM supplemented with the cytokine cocktail of: 20 ng/ml IL-3, 250ng/ml IL-6, 10 ng/ml SCF, 250 ng/ml TPO and 100 ng/ml flt-3L and a 50% mixture of LPCM. LPCM is generated by obtaining a fresh human placenta from vaginal delivery and placing it in a sterile plastic container. The placenta is rinsed with an anticoagulant solution comprising phosphate buffered saline (Gibco-Invitrogen, Grand Island, N.Y.), containing a 1:1000 concentration of heparin (1% w/w) (American Pharmaceutical Partners, Schaumburg, Ill.). The placenta is then covered with a DMEM media (Gibco) in a sterile container such that the entirety of the placenta is submerged in said media, and incubated at 37.degree. C. in a humidified 5% CO.sub.2 incubator for 24 hours. At the end of the 24 hours, the live placenta conditioned medium (LPCM) is isolated from the container and sterile-filtered using a commercially available sterile 0.2 micron filter (VWR). Cells are expanded, checked for purity using CD34-specific flow cytometry and immunologically matched to recipients using a mixed lymphocyte reaction. Cells eliciting a low level of allostimulatory activity to recipient lymphocytes are selected for transplantation. Cells are administered as described above. Patients in the treated group display an increased responsiveness to insulin starting 2 weeks after injection of cells.

### Example 2

### Increased Insulin Responsiveness After Allogeneic Endometrial Regenerative Cell

A group of 100 patients are recruited with type 2 diabetes receiving daily insulin injections. 50 patients are treated with placebo control and 50 receive allogeneic menstrual blood derived endometrial regenerative cells (ERC). ERC are generated as described in Meng et al. Endometrial regenerative cells: a novel stem cell population.

J Transl Med. 2007 Nov 15;5:57. Cells are injected intramuscularly in the gastrocnemius muscle as described in the literature (Durdu et al. J Vasc Surg. 2006 October;44(4):732-9) with a concentration of 40 million cells per limb. Patients in the treated group display an increased responsiveness to insulin starting 2 weeks after injection of cells.

One skilled in the art will appreciate that these methods, compositions, and cells are and may be adapted to carry out the objects and obtain the ends and advantages mentioned, as well as those inherent therein. The methods, procedures, and devices described herein are presently representative of preferred embodiments and are exemplary and are not intended as limitations on the scope of the invention. Changes therein and other uses will occur to those skilled in the art which are encompassed within the spirit of the invention and are defined by the scope of the disclosure. It will be apparent to one skilled in the art that varying substitutions and modifications may be made to the invention disclosed herein without departing from the scope and spirit of the invention. Those skilled in the art recognize that the aspects and embodiments of the invention set forth herein may be practiced separate from each other or in conjunction with each other. Therefore, combinations of separate embodiments are within the scope of the invention as disclosed herein. All patents and publications mentioned in the specification are indicative of the levels of those skilled in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. Thus, for example, in each instance within the description, any of the terms “comprising,” “consisting essentially of” and “consisting of” may be replaced with either of the other two terms. The terms and expressions which have been employed are used as terms of description and not of limitation, and there is no intention that in the use of such terms and expressions indicates the exclusion of equivalents of the features shown and described or portions thereof. It is recognized that various modifications are possible within the scope of the invention disclosed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the disclosure.

### REFERENCES

1. Paoletti, R., Bolego, C., Poli, A., and Cignarella, A. 2006. Metabolic syndrome, inflammation and atherosclerosis. Vasc Health Risk Manag 2:145-152.
2. Scheen, A. J. 2007. Drug-drug and food-drug pharmacokinetic interactions with new insulinotropic agents repaglinide and nateglinide. Clin Pharmacokinet 46:93-108.
3. Joshi, S. R. 2005. Metformin: old wine in new bottle—evolving technology and therapy in diabetes. J. Assoc Physicians India 53:963-972.
4. Goodarzi, M. O., and Bryer-Ash, M. 2005. Metformin revisited: re-evaluation of its properties and role in the pharmacopoeia of modern antidiabetic agents. Diabetes Obes Metab 7:654-665.
5. Eriksson, A., Attvall, S., Bonnier, M., Eriksson, J. W., Rosander, B., and Karlsson, F. A. 2007. Short-term effects of metformin in type 2 diabetes. Diabetes Obes Metab 9:330-336.
6. Green, B. D., Irwin, N., Duffy, N. A., Gault, V. A., O'Harte F, P., and Flatt, P. R. 2006. Inhibition of dipeptidyl peptidase-IV activity by metformin enhances the antidiabetic effects of glucagon-like peptide-1. Eur J Pharmacol 547:192-199.
7. Faveeuw, C., Fougeray, S., Angeli, V., Fontaine, J., Chinetti, G., Gosset, P., Delerive, P., Maliszewski, C., Capron, M., Staels, B., et al. 2000. Peroxisome proliferator-activated receptor gamma activators inhibit interleukin-12 production in murine dendritic cells. FEBS Lett 486:261-266.
8. Saubermann, L. J., Nakajima, A., Wada, K., Zhao, S., Terauchi, Y., Kadowaki, T., Aburatani, H., Matsuhashi, N., Nagai, R., and Blumberg, R. S. 2002. Peroxisome proliferator-activated receptor gamma agonist ligands stimulate a Th2 cytokine response and prevent acute colitis. Inflamm Bowel Dis 8:330-339.
9. Thomas, M. K., Rastalsky, N., Lee, J. H., and Habener, J. F. 2000. Hedgehog signaling regulation of insulin production by pancreatic beta-cells. Diabetes 49:2039-2047.
10. Oh, S. H., Muzzonigro, T. M., Bae, S. H., LaPlante, J. M., Hatch, H. M., and Petersen, B. E. 2004. Adult bone marrow-derived cells trans-differentiating into insulin-producing cells for the treatment of type I diabetes. Lab Invest 84:607-617.
11. Donath, M. Y., Ehses, J. A., Maedler, K., Schumann, D. M., Ellingsgaard, H., Eppler, E., and Reinecke, M. 2005. Mechanisms of beta-cell death in type 2 diabetes. Diabetes 54 Suppl 2:S108-113.
12. Brand, S. J., Tagerud, S., Lambert, P., Magil, S. G., Tatarkiewicz, K., Doiron, K., and Yan, Y. 2002. Pharmacological treatment of chronic diabetes by stimulating pancreatic beta-cell regeneration with systemic co-administration of EGF and gastrin. Pharmacol Toxicol 91:414-420.
13. Suarez-Pinzon, W. L., Lakey, J. R., Brand, S. J., and Rabinovitch, A. 2005. Combination therapy with epidermal growth factor and gastrin induces neogenesis of human islet {beta}-cells from pancreatic duct cells and an increase in functional {beta}-cell mass. J Clin Endocrinol Metab 90:3401-3409.
14. Suarez-Pinzon, W. L., Yan, Y., Power, R., Brand, S. J., and Rabinovitch, A. 2005. Combination therapy with epidermal growth factor and gastrin increases beta-cell mass and reverses hyperglycemia in diabetic NOD mice. Diabetes 54:2596-2601.
15. DeFronzo, R. A. 1988. Lilly lecture 1987. The triumvirate: beta-cell, muscle, liver.

A collusion responsible for NIDDM. Diabetes 37:667-687.
16. Cheetham, C., O'Driscoll, G., Stanton, K., Taylor, R., and Green, D. 2001. Losartan, an angiotensin type I receptor antagonist, improves conduit vessel endothelial function in Type II diabetes. Clin Sci (Lond) 100:13-17.
17. Kajiguchi, M., Kondo, T., Izawa, H., Kobayashi, M., Yamamoto, K., Shintani, S., Numaguchi, Y., Naoe, T., Takamatsu, J., Komori, K., et al. 2007. Safety and efficacy of autologous progenitor cell transplantation for therapeutic angiogenesis in patients with critical limb ischemia. Circ J 71:196-201.
18. Tsai, P. T., Ohab, J. J., Kertesz, N., Groszer, M., Matter, C., Gao, J., Liu, X., Wu, H., and Carmichael, S. T. 2006. A critical role of erythropoietin receptor in neurogenesis and post-stroke recovery. J Neurosci 26:1269-1274.
19. Ogueta, S., Munoz, J., Obregon, E., Delgado-Baeza, E., and Garcia-Ruiz, J. P. 2002. Prolactin is a component of the human synovial liquid and modulates the growth and chondrogenic differentiation of bone marrow-derived mesenchymal stem cells. Mol Cell Endocrinol 190:51-63.
20. Banerjee, M., Kanitkar, M., and Bhonde, R. R. 2005. Approaches towards endogenous pancreatic regeneration. Rev Diabet Stud 2:165-176.
21. Wang, C., Jiao, C., Hanlon, H. D., Zheng, W., Tomanek, R. J., and Schatteman, G. C. 2004. Mechanical, cellular, and molecular factors interact to modulate circulating endothelial cell progenitors. Am J Physiol Heart Circ Physiol 286:H1985-1993.
22. Yildirim, S., Boehmler, A. M., Kanz, L., and Mohle, R. 2005. Expansion of cord blood CD34+ hematopoietic progenitor cells in coculture with autologous umbilical vein endothelial cells (HUVEC) is superior to cytokine-supplemented liquid culture. Bone Marrow Transplant 36:71-79.
23. Ablin, J. N., Boguslayski, V., Aloush, V., Elkayam, O., Paran, D., Caspi, D., and George, J. 2006. Effect of anti-TNFalpha treatment on circulating endothelial progenitor cells (EPCs) in rheumatoid arthritis. Life Sci 79:2364-2369.
24. Bossolasco, P., Montemurro, T., Cova, L., Zangrossi, S., Calzarossa, C., Buiatiotis, S., Soligo, D., Bosari, S., Silani, V., Deliliers, G. L., et al. 2006. Molecular and phenotypic characterization of human amniotic fluid cells and their differentiation potential. Cell Res 16:329-336.
25. Sartore, S., Lenzi, M., Angelini, A., Chiavegato, A., Gasparotto, L., De Coppi, P., Bianco, R., and Gerosa, G. 2005. Amniotic mesenchymal cells autotransplanted in a porcine model of cardiac ischemia do not differentiate to cardiogenic phenotypes. Eur J Cardiothorac Surg 28:677-684.
26. Prusa, A. R., Marton, E., Rosner, M., Bettelheim, D., Lubec, G., Pollack, A., Bernaschek, G., and Hengstschlager, M. 2004. Neurogenic cells in human amniotic fluid. Am J Obstet Gynecol 191:309-314.
27. Tsai, M. S., Hwang, S. M., Tsai, Y. L., Cheng, F. C., Lee, J. L., and Chang, Y. J. 2006. Clonal amniotic fluid-derived stem cells express characteristics of both mesenchymal and neural stem cells. Biol Reprod 74:545-551.
28. Vrana, K. E., Hipp, J. D., Goss, A. M., McCool, B. A., Riddle, D. R., Walker, S. J., Wettstein, P. J., Studer, L. P., Tabar, V., Cunniff, K., et al. 2003. Nonhuman primate parthenogenetic stem cells. Proc Natl Acad Sci USA 100 Suppl 1:11911-11916.
29. Forbes, S. J., and Alison, M. R. 2006. Side population (SP) cells taking center stage in regeneration and liver cancer? Hepatology 44:23-26.
30. Meissner, K., Heydrich, B., Jedlitschky, G., Meyer Zu Schwabedissen, H., Mosyagin, I., Dazert, P., Eckel, L., Vogelgesang, S., Warzok, R. W., Bohm, M., et al. 2006. The ATP-binding cassette transporter ABCG2 (BCRP), a marker for side population stem cells, is expressed in human heart. J Histochem Cytochem 54:215-221.
31. Asakura, A., and Rudnicki, M. A. 2002. Side population cells from diverse adult tissues are capable of in vitro hematopoietic differentiation. Exp Hematol 30:1339-1345.
32. Hierlihy, A. M., Seale, P., Lobe, C. G., Rudnicki, M. A., and Megeney, L. A. 2002. The post-natal heart contains a myocardial stem cell population. FEBS Lett 530:239-243.
33. Rasmussen, B. B., Fujita, S., Wolfe, R. R., Mittendorfer, B., Roy, M., Rowe, V. L., and Volpi, E. 2006. Insulin resistance of muscle protein metabolism in aging. Faseb J 20:768-769.
34. Scheen, A. J. 2005. Diabetes mellitus in the elderly: insulin resistance and/or impaired insulin secretion? Diabetes Metab 31 Spec No 2:5S27-25S34.
35. Borst, S. E., and Bagby, G. J. 2002. Neutralization of tumor necrosis factor reverses age-induced impairment of insulin responsiveness in skeletal muscle of Sprague-Dawley rats. Metabolism 51:1061-1064.
36. Brzoska, E., Grabowska, I., Hoser, G., Streminska, W., Wasilewska, D., Machaj, E. K., Pojda, Z., Moraczewski, J., and Kawiak, J. 2006. Participation of stem cells from human cord blood in skeletal muscle regeneration of SCID mice. Exp Hematol 34:1262-1270.
37. Madeddu, P., Emanueli, C., Pelosi, E., Salis, M. B., Cerio, A. M., Bonanno, G., Patti, M., Stassi, G., Condorelli, G., and Peschle, C. 2004. Transplantation of low dose CD34+KDR+ cells promotes vascular and muscular regeneration in ischemic limbs. Faseb J 18:1737-1739.
38. Gang, E. J., Jeong, J. A., Hong, S. H., Hwang, S. H., Kim, S. W., Yang, I. H., Ahn, C., Han, H., and Kim, H. 2004. Skeletal myogenic differentiation of mesenchymal stem cells isolated from human umbilical cord blood. Stem Cells 22:617-624.
39. Oreffo, R. O., Cooper, C., Mason, C., and Clements, M. 2005. Mesenchymal stem cells: lineage, plasticity, and skeletal therapeutic potential. Stem Cell Rev 1:169-178.
40. Sacco, A., Doyonnas, R., LaBarge, M. A., Hammer, M. M., Kraft, P., and Blau, H. M. 2005. IGF-I increases bone marrow contribution to adult skeletal muscle and enhances the fusion of myelomonocytic precursors. J Cell Biol 171:483-492.
41. Dezawa, M., Ishikawa, H., Itokazu, Y., Yoshihara, T., Hoshino, M., Takeda, S., Ide, C., and Nabeshima, Y. 2005. Bone marrow stromal cells generate muscle cells and repair muscle degeneration. Science 309:314-317.
42. Lee, J. H., Kosinski, P. A., and Kemp, D. M. 2005. Contribution of human bone marrow stem cells to individual skeletal myotubes followed by myogenic gene activation. Exp Cell Res 307:174-182.
43. Palermo, A. T., Labarge, M. A., Doyonnas, R., Pomerantz, J., and Blau, H. M. 2005. Bone marrow contribution to skeletal muscle: a physiological response to stress. Dev Biol 279:336-344.
44. Sherwood, R. I., Christensen, J. L., Weissman, I. L., and Wagers, A. J. 2004. Determinants of skeletal muscle contributions from circulating cells, bone marrow cells, and hematopoietic stem cells. Stem Cells 22:1292-1304.
45. Santa Maria, L., Rojas, C. V., and Minguell, J. J. 2004. Signals from damaged but not undamaged skeletal muscle induce myogenic differentiation of rat bone-marrow-derived mesenchymal stem cells. Exp Cell Res 300:418-426.
46. Natsu, K., Ochi, M., Mochizuki, Y., Hachisuka, H., Yanada, S., and Yasunaga, Y. 2004. Allogeneic bone marrow-derived mesenchymal stromal cells promote the regeneration of injured skeletal muscle without differentiation into myofibers. Tissue Eng 10:1093-1112.
47. Doyonnas, R., LaBarge, M. A., Sacco, A., Charlton, C., and Blau, H. M. 2004. Hematopoietic contribution to skeletal muscle regeneration by myelomonocytic precursors. Proc Natl Acad Sci USA 101:13507-13512.
48. Abedi, M., Greer, D. A., Colvin, G. A., Demers, D. A., Dooner, M. S., Harpel, J. A., Weier, H. U., Lambert, J. F., and Quesenberry, P. J. 2004. Robust conversion of marrow cells to skeletal muscle with formation of marrow-derived muscle cell colonies: a multifactorial process. Exp Hematol 32:426-434.
49. Bhagavati, S., and Xu, W. 2004. Isolation and enrichment of skeletal muscle progenitor cells from mouse bone marrow. Biochem Biophys Res Commun 318:119-124.
50. Bossolasco, P., Corti, S., Strazzer, S., Borsotti, C., Del Bo, R., Fortunato, F., Salani, S., Quirici, N., Bertolini, F., Gobbi, A., et al. 2004. Skeletal muscle differentiation potential of human adult bone marrow cells. Exp Cell Res 295:66-78.
51. Musaro, A., Giacinti, C., Borsellino, G., Dobrowolny, G., Pelosi, L., Cairns, L., Ottolenghi, S., Cossu, G., Bernardi, G., Battistini, L., et al. 2004. Stem cell-mediated muscle regeneration is enhanced by local isoform of insulin-like growth factor 1. Proc Natl Acad Sci USA 101:1206-1210.
52. Brazelton, T. R., Nystrom, M., and Blau, H. M. 2003. Significant differences among skeletal muscles in the incorporation of bone marrow-derived cells. Dev Biol 262:64-74.
53. Pennisi, E. 1998. Bone marrow cells may provide muscle power. Science 279:1456.
54. Ferrari, G., Cusella-De Angelis, G., Coletta, M., Paolucci, E., Stornaiuolo, A., Cossu, G., and Mavilio, F. 1998. Muscle regeneration by bone marrow-derived myogenic progenitors. Science 279:1528-1530.
55. Kim, M., Choi, Y. S., Yang, S. H., Hong, H. N., Cho, S. W., Cha, S. M., Pak, J. H., Kim, C. W., Kwon, S. W., and Park, C. J. 2006. Muscle regeneration by adipose tissue-derived adult stem cells attached to injectable PLGA spheres. Biochem Biophys Res Commun 348:386-392.
56. Di Rocco, G., Iachininoto, M. G., Tritarelli, A., Straino, S., Zacheo, A., Germani, A., Crea, F., and Capogrossi, M. C. 2006. Myogenic potential of adipose-tissue-derived cells. J Cell Sci 119:2945-2952.
57. Lee, J. H., and Kemp, D. M. 2006. Human adipose-derived stem cells display myogenic potential and perturbed function in hypoxic conditions. Biochem Biophys Res Commun 341:882-888.
58. Zuk, P. A., Zhu, M., Mizuno, H., Huang, J., Futrell, J. W., Katz, A. J., Benhaim, P., Lorenz, H. P., and Hedrick, M. H. 2001. Multilineage cells from human adipose tissue: implications for cell-based therapies. Tissue Eng 7:211-228.
59. Edelberg, J. M., Tang, L., Hattori, K., Lyden, D., and Rafii, S. 2002. Young adult bone marrow-derived endothelial precursor cells restore aging-impaired cardiac angiogenic function. Circ Res 90:E89-93.
60. Rubinstein, P., Dobrila, L., Rosenfield, R. E., Adamson, J. W., Migliaccio, G., Migliaccio, A. R., Taylor, P. E., and Stevens, C. E. 1995. Processing and cryopreservation of placental/umbilical cord blood for unrelated bone marrow reconstitution. Proc Natl Acad Sci USA 92:10119-10122.
61. Pickup, J. C. 2004. Inflammation and activated innate immunity in the pathogenesis of type 2 diabetes. Diabetes Care 27:813-823.
62. dKern, P. A., Ranganathan, S., Li, C., Wood, L., and Ranganathan, G. 2001. Adipose tissue tumor necrosis factor and interleukin-6 expression in human obesity and insulin resistance. Am J Physiol Endocrinol Metab 280:E745-751.
63. Winkler, G., Kiss, S., Keszthelyi, L., Sapi, Z., Ory, I., Salamon, F., Kovacs, M., Vargha, P., Szekeres, O., Speer, G., et al. 2003. Expression of tumor necrosis factor (TNF)-alpha protein in the subcutaneous and visceral adipose tissue in correlation with adipocyte cell volume, serum TNF-alpha, soluble serum TNF-receptor-2 concentrations and C-peptide level. Eur J Endocrinol 149:129-135.
64. Krogh-Madsen, R., Plomgaard, P., Moller, K., Mittendorfer, B., and Pedersen, B. K. 2006. Influence of TNF-alpha and IL-6 infusions on insulin sensitivity and expression of IL-18 in humans. Am J Physiol Endocrinol Metab 291:E108-114.
65. Derosa, G., Cicero, A. F., D'Angelo, A., Ragonesi, P. D., Ciccarelli, L., Piccinni, M. N., Pricolo, F., Salvadeo, S. A., Ferrari, I., Gravina, A., et al. 2006. Telmisartan and irbesartan therapy in type 2 diabetic patients treated with rosiglitazone: effects on insulin-resistance, leptin and tumor necrosis factor-alpha. Hypertens Res 29:849-856.
66. Hotamisligil, G. S., Peraldi, P., Budavari, A., Ellis, R., White, M. F., and Spiegelman, B. M. 1996. IRS-1-mediated inhibition of insulin receptor tyrosine kinase activity in TNF-alpha- and obesity-induced insulin resistance. Science 271:665-668.
67. Ofei, F., Hurel, S., Newkirk, J., Sopwith, M., and Taylor, R. 1996. Effects of an engineered human anti-TNF-alpha antibody (CDP571) on insulin sensitivity and glycemic control in patients with NIDDM. Diabetes 45:881-885.
68. Dominguez, H., Storgaard, H., Rask-Madsen, C., Steffen Hermann, T., Ihlemann, N., Baunbjerg Nielsen, D., Spohr, C., Kober, L., Vaag, A., and Torp-Pedersen, C. 2005. Metabolic and vascular effects of tumor necrosis factor-alpha blockade with etanercept in obese patients with type 2 diabetes. J Vasc Res 42:517-525.
69. Moschen, A. R., Kaser, A., Enrich, B., Mosheimer, B., Theurl, M., Niederegger, H., and Tilg, H. 2007. Visfatin, an adipocytokine with proinflammatory and immunomodulating properties. J Immunol 178:1748-1758.
70. Janowska, J., Zahorska-Markiewicz, B., and Olszanecka-Glinianowicz, M. 2006. Relationship between serum resistin concentration and proinflammatory cytokines in obese women with impaired and normal glucose tolerance. Metabolism 55:1495-1499.
71. Tam, L. S., Tomlinson, B., Chu, T. T., Li, T. K., and Li, E. K. 2007. Impact of TNF inhibition on insulin resistance and lipids levels in patients with rheumatoid arthritis. Clin Rheumatol.
72. Deng, W., Han, Q., Liao, L., Li, C., Ge, W., Zhao, Z., You, S., Deng, H., and Zhao, R. C. 2004. Allogeneic bone marrow-derived flk-1+Sca-1-mesenchymal stem cells leads to stable mixed chimerism and donor-specific tolerance. Exp Hematol 32:861-867.
73. Kadri, T., Lataillade, J. J., Doucet, C., Marie, A., Ernou, I., Bourin, P., Joubert-Caron, R., Caron, M., and Lutomski, D. 2005. Proteomic study of Galectin-1 expression in human mesenchymal stem cells. Stem Cells Dev 14:204-212.
74. Ryan, J. M., Barry, F. P., Murphy, J. M., and Mahon, B. P. 2005. Mesenchymal stem cells avoid allogeneic rejection. J Inflamm (Lond) 2:8.
75. Beyth, S., Borovsky, Z., Mevorach, D., Liebergall, M., Gazit, Z., Aslan, H., Galun, E., and Rachmilewitz, J. 2005. Human mesenchymal stem cells alter antigen-presenting cell maturation and induce T-cell unresponsiveness. Blood 105:2214-2219.
76. Aggarwal, S., and Pittenger, M. F. 2005. Human mesenchymal stem cells modulate allogeneic immune cell responses. Blood 105:1815-1822.
77. Cai, D., Yuan, M., Frantz, D.F., Melendez, P.A., Hansen, L., Lee, J., and Shoelson, S. E. 2005. Local and systemic insulin resistance resulting from hepatic activation of IKK-beta and NF-kappaB. Nat Med 11:183-190.
78. Plumas, J., Chaperot, L., Richard, M. J., Molens, J. P., Bensa, J. C., and Favrot, M. C. 2005. Mesenchymal stem cells induce apoptosis of activated T cells. Leukemia 19:1597-1604.
79. Maccario, R., Podesta, M., Moretta, A., Cometa, A., Comoli, P., Montagna, D., Daudt, L., Ibatici, A., Piaggio, G., Pozzi, S., et al. 2005. Interaction of human mesenchymal stem cells with cells involved in alloantigen-specific immune response favors the differentiation of CD4+T-cell subsets expressing a regulatory/suppressive phenotype. Haematologica 90:516-525.
80. Zappia, E., Casazza, S., Pedemonte, E., Benvenuto, F., Bonanni, I., Gerdoni, E., Giunti, D., Ceravolo, A., Cazzanti, F., Frassoni, F., et al. 2005. Mesenchymal stem cells ameliorate experimental autoimmune encephalomyelitis inducing T-cell anergy. Blood 106:1755-1761.

What is claimed is:

1. A method of ameliorating the effects of insulin resistance in a mammal comprising: identifying a mammal suffering from insulin resistance; and administering to said mammal a mesenchymal stem cell population in an amount sufficient to increase insulin sensitivity in the mammal and wherein the mesenchymal stem cell population expresses the markers CD90 and CD105 while substantially lacking CD45 and CD14 expression.

2. The method of claim 1, wherein the insulin resistance is associated with Type 2 diabetes.

3. The method of claim 1, wherein said mesenchymal stem cells are derived from bone marrow and are mononuclear cells.

4. The method of claim 3 wherein said bone marrow cells are administered at a concentration of approximately 1-3 billion mononuclear cells.

5. The method of claim 1, wherein said insulin resistance is measured using the hyperinsulinemic euglycemia clamp technique.

6. The method of claim 1, wherein said insulin resistance is defined by an abnormal reading on a series of tests selected from the group consisting of: IV glucose tolerance test (FSIVGTT), insulin tolerance test (ITT), insulin sensitivity test (IST), the continuous infusion of glucose with model assessment (CIGMA), and the oral glucose tolerance test (OGTT).

7. The method of claim 1, wherein said mesenchymal stem cell population possesses anti-inflammatory properties.

8. The method of claim 1, wherein said mesenchymal stem cell population is capable of adhering to a tissue culture vessel.

9. The method of claim 1, wherein said mesenchymal stem cells are endometrial regenerative cells (ERCs).

10. The method of claim 9, wherein said ERCs are administered at a concentration of approximately 40 million cells.

11. The method of claim 9, wherein said insulin resistance is defined by an abnormal reading on the hyperinsulinemic euglycemia clamp.

12. The method of claim 9, wherein said insulin resistance is defined by an abnormal reading on a series of tests selected from the group consisting of: IV glucose tolerance test (FSIVGTT), insulin tolerance test (ITT), insulin sensitivity test (IST), the continuous infusion of glucose with model assessment (CIGMA), and the oral glucose tolerance test (OGTT).

13. The method of claim 9, wherein said ERCs possesses anti-inflammatory properties.

14. The method of claim 9, wherein said ERCs are capable of adhering to a tissue culture vessel.
